(19) United States
(12) Patent Application Publication
(10) Pub. No.: US 2015/0328220 A1
(43) Pub. Date: Nov. 19, 2015

(54) METHODS OF TREATING CHRONIC KIDNEY DISEASE CHARACTERIZED BY MACROALBUMINURIA

(71) Applicant: Concert Pharmaceuticals, Inc., Lexington, MA (US)

(72) Inventor: Philip B. Graham, Carlisle, MA (US)

(21) Appl. No.: 14/711,922

(22) Filed: May 14, 2015

Related U.S. Application Data
(60) Provisional application No. 62/023,521, filed on Jul. 11, 2014.
(60) Provisional application No. 61/993,093, filed on May 14, 2014.

(51) Int. Cl. A61K 31/522

(52) U.S. Cl. CPC A61K 31/522

(57) ABSTRACT

The present invention relates to a novel method of treating chronic kidney disease in a patient with macroalbuminuria. The method comprises administering to the patient an effective amount of a compound described herein.

### RELATED APPLICATIONS

This application claims the benefit of U.S. Provisional Application Ser. No. 61/993,093, filed May 14, 2014 and also claims the benefit of U.S. Provisional Application Ser. No. 62/023,521, filed Jul. 11, 2014. The contents of these applications are incorporated herein by reference in their entirety.

### BACKGROUND OF THE INVENTION

Chronic kidney disease (CKD) is a condition characterized by a gradual loss of kidney function over time. Kidney disease may eventually lead to kidney failure, which requires dialysis or a kidney transplant to maintain life. According to the National Kidney Foundation, 26 million Americans have CKD and millions of others are at increased risk. The two main causes of chronic kidney disease are diabetes and high blood pressure, which are responsible for up to two-thirds of the cases. Other conditions that affect the kidneys include glomerulonephritis, a group of diseases that cause inflammation and damage to the kidney's filtering units; inherited diseases, such as polycystic kidney disease, which causes large cysts to form in the kidneys and damages the surrounding tissue; malformations that occur in utero; lupus and other diseases that affect the body's immune system; obstructions caused by problems like kidney stones, tumors or an enlarged prostate gland in men; poisonous agents or medications that are taken in doses or over chronic durations that exceed safety parameters, or are taken concomitantly with other medications that are contraindicated due to safety risks; environmental exposure; physical trauma, for example, direct and forceful blow to the kidneys and repeated urinary infections.

Kidney function is best estimated by glomerular filtration rate (GFR) and usually expressed as estimated GFR or eGFR. This is typically calculated by measuring levels of serum creatinine (measured in mg/dL), a waste product in the blood, and then applying a mathematical formula that accounts for additional variables, including age, race and gender. If the filtration in the kidney is deficient, serum creatinine levels are higher than normal; increasing serum creatinine indicates a worsening of kidney function. GFR and eGFR are measured in units of milliliters per minute per 1.73 meters squared, which is a typical adult body surface area. Chromic kidney disease is defined as significant loss of kidney function as measured by an eGFR of <60 for three months or evidence of kidney damage such as persistent proteinuria or pathological abnormalities from biopsy or imaging tests. Chronic kidney disease is categorized by stages to guide clinical practice. Stage 1 is an eGFR of >90 considered normal or increased eGFR with evidence of kidney damage; Stage 2 is eGFR 60 to 89 with evidence of kidney damage known as mild or early stage kidney disease, Stage 3 is 30 to 59 or moderate or mid-stage kidney disease, Stage 4 is 15 to 29 or severe or advanced kidney disease and Stage 5 is below 15 which indicates kidney failure and if sustained or worsens, requires dialysis or kidney transplantation to avoid death.

“Proteinuria” refers to a condition where protein from blood leaks in excess (typically at least 500 mg per day) via damaged renal epithelium into the patient's urine. Sustained proteinuria is a sign of chronic kidney disease (CKD) regardless of GFR. Albumin is a protein normally found in the blood and prevented from passing into urine when the kidneys are healthy. Albumin is a smaller protein molecule and is often the first protein to be detected in urine with damage to the glomeruli and in the presence of diabetes and hypertension. “Albuminuria” may go unnoticed in its early signs, and if left untreated, may increase over time to macroalbuminuria (>300 mg/day) and accelerate the progression of kidney disease. This progression occurs through multiple pathways, resulting in tubulointerstitial injury and fibrosis whereby albumin leaks into the urine via the damaged epithelium. Like proteinuria, a persistent high level of albumin, or albuminuria, is therefore also evidence of kidney damage.

The typical measurement for albuminuria is the ratio of urine albumin to urine creatinine, or UACR, expressed as milligrams of albumin per gram of creatinine. A UACR greater than 300 mg/g is referred to as macroalbuminuria and, when sustained for three months or longer, generally indicates substantial kidney damage.

Current standard of care for chronic kidney disease is aimed at renoprotective effects, such as proteinuria reduction and decreased rate of GFR decline, by using treatment with angiotensin modulators, particularly the angiotensin-converting enzyme (ACE) inhibitors and the angiotensin II receptor blockers (ARBs). These are antihypertensive agents that also have the effect of reducing albuminuria and slowing the decline of renal function. However, despite treatment with these drugs, many chronic kidney disease patients continue to experience loss in renal function at a rate that is significantly faster than normal age-related decline. No new disease modifying treatments for chronic kidney disease have been approved by the FDA in the last decade.

Chronic kidney disease is a major health problem in today's society and available treatments are only partially effective. There is a great need for new medicines to treat CKD.

### SUMMARY OF THE INVENTION

We have found that, relative to placebo, a deuterated analog of 1-(S)-5-hydroxyhexyl-3,7-dimethylxanthine can slow the progression of chronic kidney disease in patients with type 2 diabetes who have macroalbuminuria. In particular, we have found that the benefits of Compound V relative to placebo are significantly greater for patients with macroalbuminuria with UACR levels above 850 mg/g. A 48 week clinical trial was conducted comparing Compound V and placebo in patients with type 2 diabetes and macroalbuminuria. In the clinical trial, it was found that fewer patients receiving Compound V experienced an undesirable, large increase in serum creatinine levels relative to patients who received placebo. The improvement in serum creatinine over 48 weeks for Compound V versus placebo was much more pronounced in those patients who had UACR levels above 850 mg/g, which was the median UACR for the clinical trial population.

This invention relates to a method of treating chronic kidney disease characterized by macroalbuminuria in a patient comprising administering to the patient an effective amount of a compound represented by the following structural formula:

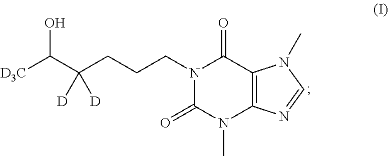 [SMILES: [2H]C([2H])([2H])C(O)C([2H])([2H])CCCn1c(=O)c2c(ncn2C)n(C)c1=O]

or a pharmaceutically acceptable salt thereof. One or more hydrogen atoms in the compound of formula (I) are optionally replaced with deuterium.

The present invention also relates to a method of treating chronic kidney disease characterized by macroalbuminuria in a patient comprising the steps of:

(a) assessing the patient's UACR; and

(b) if the patient's UACR is greater than a threshold, the threshold level being 500 mg/g, administering to the patient an effective amount of a compound of structural formula (I) or a pharmaceutically acceptable salt thereof. In some embodiments, the threshold level is 500 mg/g, but not greater than 5000 mg/g.

The present invention also relates to a method of treating chronic kidney disease characterized by proteinuria in a patient comprising the steps of:

(a) assessing the patient's protein to creatinine ratio (PCR); and

(b) if the patient's PCR is greater than a threshold, the threshold level being 700 mg/g, administering to the patient an effective amount of a compound of structural formula (I) or a pharmaceutically acceptable salt thereof. In some embodiments, the threshold level is 700 mg/g, but not greater than 8500 mg/g.

### BRIEF DESCRIPTION OF THE FIGURES

FIG. 1 is a graph showing the amount of urinary fibronectin in patients treated with Compound (V) or a placebo following a 48 week clinical trial.

FIG. 2 is a graph showing the amount of urinary fibronectin in patients with (a) UACR greater than 850 mg/g treated with Compound (V) or a placebo following a 48 week clinical trial and (b) UACR less than 850 mg/g treated with Compound (V) or a placebo following a 48 week clinical trial.

FIG. 3 is a graph showing the amount of plasma collagen in patients treated with Compound (V) or a placebo following a 48 clinical trial.

### DETAILED DESCRIPTION OF THE INVENTION

The invention provides a method of treating chronic kidney disease characterized by macroalbuminuria in a patient comprising administering to the patient an effective amount of a compound of structural formula (I) or a pharmaceutically acceptable salt thereof.

An individual is classified as having chronic kidney disease if he or she has a GFR of less than 60 mL/min/1.73 m² for at least three months or evidence of kidney damage such as persistent proteinuria or pathological abnormalities from biopsy or imaging tests. The invention also provides a method of treating conditions that are associated with chronic kidney disease or affect kidney function comprising administering to the patient an effective amount of a compound of structural formula (I) or a pharmaceutically acceptable salt thereof. The chronic kidney disease treatable by the disclosed methods may be associated with chronic hyperglycemia. While the clinical trial reported herein provides supportive proof of concept, particularly in type 2 diabetic kidney disease patients, patients with type 1 (insulin-dependent) diabetic kidney disease are also at increased risk of declining glomerular filtration rate. Conditions treatable by the disclosed methods also include, for example, primary glomerular diseases (for example, membranous glomerulonephritis, immunoglobulin A (IgA) nephropathy, focal segmental glomerulosclerosis, and membranoproliferative glomerulonephritis); Secondary glomerular diseases (for example, hypertensive nephropathy, diabetic nephropathy, reflux nephropathy, lupus nephritis, microscopic polyangitis, granulomatous polyangitis (Wegener's granulomatosis), and anti-glomerular basement membrane antibody disease (Goodpasture's disease)), obstructions from kidney stones, benign prostatic hypertrophy and tumors, inherited diseases such as, polycystic kidney disease; and Tubulointerstitial diseases (for example, idiopathic chronic interstitial nephritis, sickle cell nephropathy, sarcoidosis, scleroderma, sjogren's disease, analgesic nephropathy, uric acid nephropathy, oxalate nephropathy), reflux nephropathy, malformations related to congenital anomalies, direct trauma to kidneys, ingestion or exposure of toxins (for example, chemical or medicinal agents) or environmental conditions (for example, heavy metals, elevated ambient temperatures, infections and the like).

In another embodiment, the patient is concomitantly treated with an angiotensin-converting enzyme inhibitor or an angiotensin receptor blocker. Angiotensin-converting enzyme inhibitors and angiotensin receptor blockers are described below. Angiotensin-converting enzyme inhibitor and angiotensin receptor blockers are approved for the treatment of kidney disease resulting from diabetes.

“Macroalbuminuria” refers to conditions where albumin excreted in a patient's urine is greater than 300 mg in a 24 hour period or is greater than 300 mg/L in a spot sample or where the UACR is greater than 300 mg albumin/g creatinine. In the disclosed methods, albumin excreted in a patient's urine in a 24 hour period is greater than about 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg or 1500 mg. Alternatively in the disclosed methods, albumin excreted in a patient's urine in a spot sample is greater than about 500 mg/L, 550 mg/L, 600 mg/L, 650 mg/L, 700 mg/L, 750 mg/L, 800 mg/L, 850 mg/L, 900 mg/L, 950 mg/L, 1000 mg/L, 1100 mg/L, 1200 mg/L, 1300 mg/L, 1400 mg/L or 1500 mg/L. Alternatively in the disclosed methods, the UACR (mg albumin/g creatinine) is greater than about 500 mg/g, 550 mg/g, 600 mg/g, 650 mg/g, 700 mg/g, 750 mg/g, 800 mg/g, 850 mg/g, 900 mg/g, 950 mg/g, 1000 mg/g, 1100 mg/g, 1200 mg/g, 1300 mg/g, 1400 mg/g or 1500 mg/g.

Proteinuria may be detected by a dipstick test in which the result with a positive dipstick is >1+ which is equivalent to about 100 mg/dL. Per NKF KDOQI Guidelines, quantitative measures such as PCR within 3 months, or UACR, is recommended for diagnosis. More than 500 mg of protein in a 24 hour urine collection can be used to confirm proteinuria or by comparing the amount of urine protein in the sample against its concentration of urine creatinine, termed the protein to creatinine ratio (PCR) in which the protein/creatinine ratio greater about 700 mg/g. In one embodiment, the PCR in the patient being treated with the present methods is greater about 700 mg/g, 800 mg/g, 900 mg/g, 1000 mg/g, 1100 mg/g, 1200 mg/g, 1300 mg/g, 1400 mg/g, 1500 mg/g, 1600 mg/g, 1700 mg/g, 1800 mg/g, 1900 mg/g, 2000 mg/g, 2100 mg/g, 2200 mg/g or 2300 mg/g.

The present invention also relates to a method of treating chronic kidney disease characterized by macroalbuminuria in a patient comprising the steps of:

(a) assessing the patient's UACR;

(b) if the patient's UACR is greater than a threshold, the threshold level being 500 mg/g, administering to the patient an effective amount of a compound of structural formula (I) or a pharmaceutically acceptable salt thereof. In some embodiments, the threshold level is 500 mg/g, but not greater than 5000 mg/g.

In one embodiment, the UACR threshold level is about 500 mg/g, 550 mg/g, 600 mg/g, 650 mg/g, 700 mg/g, 750 mg/g, 800 mg/g, 850 mg/g, 900 mg/g, 950 mg/g, 1000 mg/g, 1100 mg/g, 1200 mg/g, 1300 mg/g, 1400 mg/g or 1500 mg/g. In one embodiment, the UACR threshold level is about 500 mg/g, 550 mg/g, 600 mg/g, 650 mg/g, 700 mg/g, 750 mg/g, 800 mg/g, 850 mg/g, 900 mg/g, 950 mg/g, 1000 mg/g, 1100 mg/g, 1200 mg/g, 1300 mg/g, 1400 mg/g or 1500 mg/g, but not greater than 5000 mg/g. In some embodiments, the patient also has type 2 diabetes.

The present invention also relates to a method of treating chronic kidney disease characterized by macroalbuminuria in a patient comprising the steps of:

(a) assessing the patient's PCR; and

(b) if the patient's PCR is greater than a threshold, the threshold level being 700 or 800 mg/g, administering to the patient an effective amount of a compound of structural formula (I) or a pharmaceutically acceptable salt thereof. In some embodiments, the threshold level is 700 or 800 mg/g but not greater than 8500 mg/g.

In one embodiment, the PCR threshold level is about 700 mg/g, 800 mg/g, 900 mg/g, 1000 mg/g, 1100 mg/g, 1200 mg/g, 1300 mg/g, 1400 mg/g, 1500 mg/g, 1600 mg/g, 1700 mg/g, 1800 mg/g, 1900 mg/g, 2000 mg/g, 2100 mg/g, 2200 mg/g or 2300 mg/g. In one embodiment, the PCR threshold level is about 700 mg/g, 800 mg/g, 900 mg/g, 1000 mg/g, 1100 mg/g, 1200 mg/g, 1300 mg/g, 1400 mg/g, 1500 mg/g, 1600 mg/g, 1700 mg/g, 1800 mg/g, 1900 mg/g, 2000 mg/g, 2100 mg/g, 2200 mg/g or 2300 mg/g, but not greater than 8500 mg/g. In some embodiments, the patient also has type 2 diabetes.

Another embodiment of the invention is a method of treating chronic kidney disease (CKD) in a patient with CKD characterized by macroalbuminuria comprising the steps of:

(a) assessing the patient's urine albumin to creatinine ratio (UACR);

(b) if the patient's UACR is greater than a threshold, the threshold level being 500 mg/g, administering to the patient an effective amount of a compound represented by structural formula (I):

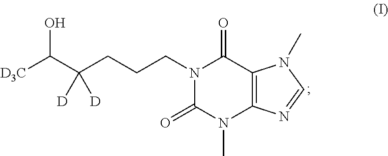 [SMILES: [2H]C([2H])([2H])C(O)C([2H])([2H])CCCn1c(=O)c2c(ncn2C)n(C)c1=O]

or a pharmaceutically acceptable salt thereof, wherein one or more hydrogen atoms are optionally replaced with deuterium; and

c) if the patient's UACR is less than the threshold, the threshold level being 500 mg/g, administering to the patient an effective amount of a chronic kidney disease therapy that does not include the compound of structural formula (I) or a pharmaceutically acceptable salt thereof. In some embodiments, the threshold level is 500 mg/g, but not greater than 5000 mg/g. Alternatively, the UACR threshold level is about 550 mg/g, 600 mg/g, 650 mg/g, 700 mg/g, 750 mg/g, 800 mg/g, 850 mg/g, 900 mg/g, 950 mg/g, 1000 mg/g, 1100 mg/g, 1200 mg/g, 1300 mg/g, 1400 mg/g or 1500 mg/g. Alternatively, the UACR threshold level is about 550 mg/g, 600 mg/g, 650 mg/g, 700 mg/g, 750 mg/g, 800 mg/g, 850 mg/g, 900 mg/g, 950 mg/g, 1000 mg/g, 1100 mg/g, 1200 mg/g, 1300 mg/g, 1400 mg/g or 1500 mg/g, but not greater than 5000 mg/g. In some embodiments, the patient also has type 2 diabetes.

Another embodiment of the invention is a method of treating chronic kidney disease (CKD) in a patient with CKD characterized by macroalbuminuria comprising the steps of:

(a) assessing the patient's protein to creatinine ratio (PCR);

(b) if the patient's PCR is greater than a threshold, the threshold level being 700 mg/g, administering to the patient an effective amount of a compound represented by structural formula (I):

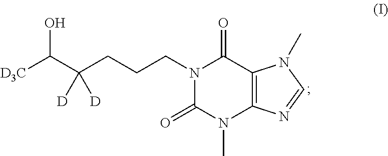 [SMILES: [2H]C([2H])([2H])C(O)C([2H])([2H])CCCn1c(=O)c2c(ncn2C)n(C)c1=O]

or a pharmaceutically acceptable salt thereof, wherein one or more hydrogen atoms are optionally replaced with deuterium; and

c) if the patient's PCR is less than the threshold, the threshold level being 700 mg/g, administering to the patient an effective amount of a chronic kidney disease therapy that does not include the compound of structural formula (I) or a pharmaceutically acceptable salt thereof. In some embodiments, the threshold level is 700 mg/g but not greater than 8500 mg/g. In another alternative, the patient is administered an effective amount of the compound of structural formula (I) if the PCR is above a threshold and a chronic kidney disease therapy that does not include the compound of structural formula (I) if the PCR is below the threshold, the threshold being about 700 mg/g, 800 mg/g, 900 mg/g, 1000 mg/g, 1100 mg/g, 1200 mg/g, 1300 mg/g, 1400 mg/g, 1500 mg/g, 1600 mg/g, 1700 mg/g, 1800 mg/g, 1900 mg/g, 2000 mg/g, 2100 mg/g, 2200 mg/g or 2300 mg/g. Alternatively, the PCR threshold level is about 700 mg/g, 800 mg/g, 900 mg/g, 1000 mg/g, 1100 mg/g, 1200 mg/g, 1300 mg/g, 1400 mg/g, 1500 mg/g, 1600 mg/g, 1700 mg/g, 1800 mg/g, 1900 mg/g, 2000 mg/g, 2100 mg/g, 2200 mg/g or 2300 mg/g, but not greater than 8500 mg/g. In some embodiments, the patient also has type 2 diabetes.

Another embodiment of the invention is a compound represented by structural formula (I)

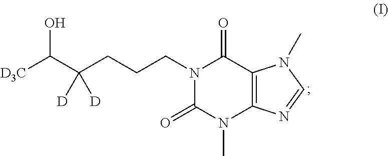 [SMILES: [2H]C([2H])([2H])C(O)C([2H])([2H])CCCn1c(=O)c2c(ncn2C)n(C)c1=O]

or a pharmaceutically acceptable salt thereof, wherein one or more hydrogen atoms are optionally replaced with deuterium, for use in treating chronic kidney disease (CKD) in a patient with CKD, wherein the patient's urine albumin to creatinine ratio (UACR) has been assessed and the compound is for administration when the UACR threshold has been assessed to be greater than a threshold, the threshold being greater than 500 mg/g. The UACR has been determined from a urine sample obtained from the patient. In some embodiments, the threshold level is 500 mg/g, but not greater than 5000 mg/g. Alternatively, the UACR threshold level is about 550 mg/g, 600 mg/g, 650 mg/g, 700 mg/g, 750 mg/g, 800 mg/g, 850 mg/g, 900 mg/g, 950 mg/g, 1000 mg/g, 1100 mg/g, 1200 mg/g, 1300 mg/g, 1400 mg/g or 1500 mg/g. Alternatively, the UACR threshold level is about 550 mg/g, 600 mg/g, 650 mg/g, 700 mg/g, 750 mg/g, 800 mg/g, 850 mg/g, 900 mg/g, 950 mg/g, 1000 mg/g, 1100 mg/g, 1200 mg/g, 1300 mg/g, 1400 mg/g or 1500 mg/g, but not greater than 5000 mg/g. In some embodiments, the patient also has type 2 diabetes.

Another embodiment of the invention is a therapeutic composition for use in treating chronic kidney disease (CKD) in a patient with CKD, wherein the patient's urine albumin to creatinine ratio (UACR) has been assessed, wherein the composition comprises a compound represented by structural formula (I)

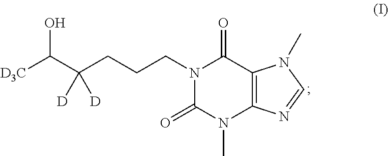 [SMILES: [2H]C([2H])([2H])C(O)C([2H])([2H])CCCn1c(=O)c2c(ncn2C)n(C)c1=O]

or a pharmaceutically acceptable salt thereof, wherein one or more hydrogen atoms are optionally replaced with deuterium, for administration when the UACR threshold has been assessed to be greater than a threshold, the threshold being greater than 500 mg/g and wherein the composition for administration does not comprise a compound represented by structural formula (I) for administration when the UACR threshold has been assessed as less than 500 mg/g (e.g., comprises a drug effective for the treatment of chronic kidney disease other than the compound represented by structural formula (I) or a pharmaceutically acceptable salt thereof). The UACR has been determined from a urine sample obtained from the patient. In some embodiments, the threshold level is 500 mg/g, but not greater than 5000 mg/g. Alternatively, the UACR threshold level is about 550 mg/g, 600 mg/g, 650 mg/g, 700 mg/g, 750 mg/g, 800 mg/g, 850 mg/g, 900 mg/g, 950 mg/g, 1000 mg/g, 1100 mg/g, 1200 mg/g, 1300 mg/g, 1400 mg/g or 1500 mg/g. Alternatively, the UACR threshold level is about 550 mg/g, 600 mg/g, 650 mg/g, 700 mg/g, 750 mg/g, 800 mg/g, 850 mg/g, 900 mg/g, 950 mg/g, 1000 mg/g, 1100 mg/g, 1200 mg/g, 1300 mg/g, 1400 mg/g or 1500 mg/g, but not greater than 5000 mg/g. In some embodiments, the patient also has type 2 diabetes.

Another embodiment of the invention is a compound represented by structural formula (I)

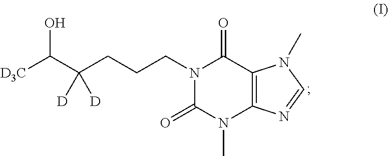 [SMILES: [2H]C([2H])([2H])C(O)C([2H])([2H])CCCn1c(=O)c2c(ncn2C)n(C)c1=O]

or a pharmaceutically acceptable salt thereof, wherein one or more hydrogen atoms are optionally replaced with deuterium, for use in treating chronic kidney disease (CKD) in a patient with CKD, wherein the patient's protein to creatinine ratio (PCR) has been assessed and the compound is for administration when the PCR threshold has been assessed to be greater than a threshold, the threshold being greater than 700 mg/g. The PCR has been determined from a urine sample obtained from the patient. In some embodiments, the threshold level is 700 mg/g but not greater than 8500 mg/g. Alternatively, the PCR threshold level is about 700 mg/g, 800 mg/g, 900 mg/g, 1000 mg/g, 1100 mg/g, 1200 mg/g, 1300 mg/g, 1400 mg/g, 1500 mg/g, 1600 mg/g, 1700 mg/g, 1800 mg/g, 1900 mg/g, 2000 mg/g, 2100 mg/g, 2200 mg/g or 2300 mg/g. Alternatively, the PCR threshold level is about 700 mg/g, 800 mg/g, 900 mg/g, 1000 mg/g, 1100 mg/g, 1200 mg/g, 1300 mg/g, 1400 mg/g, 1500 mg/g, 1600 mg/g, 1700 mg/g, 1800 mg/g, 1900 mg/g, 2000 mg/g, 2100 mg/g, 2200 mg/g or 2300 mg/g, but not greater than 8500 mg/g. In some embodiments, the patient also has type 2 diabetes.

Another embodiment of the invention is a therapeutic composition for use in treating chronic kidney disease (CKD) in a patient with CKD, wherein the patient's protein to creatinine ratio (PCR) has been assessed, wherein the composition comprises a compound represented by structural formula (I)

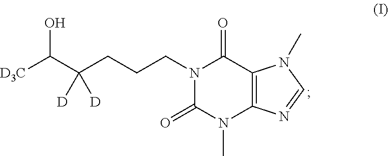 [SMILES: [2H]C([2H])([2H])C(O)C([2H])([2H])CCCn1c(=O)c2c(ncn2C)n(C)c1=O]

or a pharmaceutically acceptable salt thereof, wherein one or more hydrogen atoms are optionally replaced with deuterium, for administration when the PCR threshold has been assessed to be greater than a threshold, the threshold being greater than 700 mg/g and wherein the composition for administration does not comprise a compound represented by structural formula (I) for administration when the PCR threshold has been assessed as less than 700 mg/g (e.g., comprises a drug effective for the treatment of chronic kidney disease other than the compound represented by structural formula (I) or a pharmaceutically acceptable salt thereof). The PCR has been determined from a urine sample obtained from the patient. In some embodiments, the threshold level is 700 mg/g but not greater than 8500 mg/g. Alternatively, the PCR threshold level is about 700 mg/g, 800 mg/g, 900 mg/g, 1000 mg/g, 1100 mg/g, 1200 mg/g, 1300 mg/g, 1400 mg/g, 1500 mg/g, 1600 mg/g, 1700 mg/g, 1800 mg/g, 1900 mg/g, 2000 mg/g, 2100 mg/g, 2200 mg/g or 2300 mg/g. Alternatively, the PCR threshold level is about 700 mg/g, 800 mg/g, 900 mg/g, 1000 mg/g, 1100 mg/g, 1200 mg/g, 1300 mg/g, 1400 mg/g, 1500 mg/g, 1600 mg/g, 1700 mg/g, 1800 mg/g, 1900 mg/g, 2000 mg/g, 2100 mg/g, 2200 mg/g or 2300 mg/g, but not greater than 8500 mg/g. In some embodiments, the patient also has type 2 diabetes.

Another embodiment of the invention is the use of a compound represented by structural formula (I)

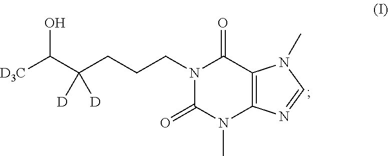 [SMILES: [2H]C([2H])([2H])C(O)C([2H])([2H])CCCn1c(=O)c2c(ncn2C)n(C)c1=O]

or a pharmaceutically acceptable salt thereof, wherein one or more hydrogen atoms are optionally replaced with deuterium, for the manufacture of a medicament for treating chronic kidney disease (CKD) in a patient with CKD, wherein the patient's urine albumin to creatinine ratio (UACR) has been assessed and the compound is for administration when the UACR threshold has been assessed to be greater than a threshold, the threshold being greater than 500 mg/g. The UACR has been determined from a urine sample obtained from the patient. In some embodiments, the threshold level is 500 mg/g, but not greater than 5000 mg/g. Alternatively, the UACR threshold level is about 550 mg/g, 600 mg/g, 650 mg/g, 700 mg/g, 750 mg/g, 800 mg/g, 850 mg/g, 900 mg/g, 950 mg/g, 1000 mg/g, 1100 mg/g, 1200 mg/g, 1300 mg/g, 1400 mg/g or 1500 mg/g. Alternatively, the UACR threshold level is about 550 mg/g, 600 mg/g, 650 mg/g, 700 mg/g, 750 mg/g, 800 mg/g, 850 mg/g, 900 mg/g, 950 mg/g, 1000 mg/g, 1100 mg/g, 1200 mg/g, 1300 mg/g, 1400 mg/g or 1500 mg/g, but not greater than 5000 mg/g. In some embodiments, the patient also has type 2 diabetes.

Another embodiment of the invention is the use of a therapeutic composition for the manufacture of a medicament for treating chronic kidney disease (CKD) in a patient with CKD, wherein the patient's urine albumin to creatinine ratio (UACR) has been assessed, wherein the composition comprises a compound represented by structural formula (I)

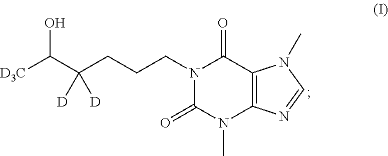 [SMILES: [2H]C([2H])([2H])C(O)C([2H])([2H])CCCn1c(=O)c2c(ncn2C)n(C)c1=O]

or a pharmaceutically acceptable salt thereof, wherein one or more hydrogen atoms are optionally replaced with deuterium, for administration when the UACR threshold has been assessed to be greater than a threshold, the threshold being greater than 500 mg/g and wherein the composition for administration does not comprise a compound represented by structural formula (I) for administration when the UACR threshold has been assessed as less than 500 mg/g (e.g., comprises a drug effective for the treatment of chronic kidney disease other than the compound represented by structural formula (I) or a pharmaceutically acceptable salt thereof). The UACR has been determined from a urine sample obtained from the patient. In some embodiments, the threshold level is 500 mg/g, but not greater than 5000 mg/g. Alternatively, the UACR threshold level is about 550 mg/g, 600 mg/g, 650 mg/g, 700 mg/g, 750 mg/g, 800 mg/g, 850 mg/g, 900 mg/g, 950 mg/g, 1000 mg/g, 1100 mg/g, 1200 mg/g, 1300 mg/g, 1400 mg/g or 1500 mg/g. Alternatively, the UACR threshold level is about 550 mg/g, 600 mg/g, 650 mg/g, 700 mg/g, 750 mg/g, 800 mg/g, 850 mg/g, 900 mg/g, 950 mg/g, 1000 mg/g, 1100 mg/g, 1200 mg/g, 1300 mg/g, 1400 mg/g or 1500 mg/g, but not greater than 5000 mg/g. In some embodiments, the patient also has type 2 diabetes.

Another embodiment of the invention is the use of a compound represented by structural formula (I)

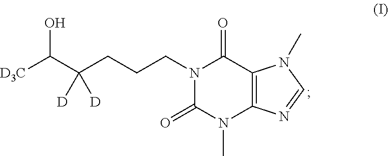 [SMILES: [2H]C([2H])([2H])C(O)C([2H])([2H])CCCn1c(=O)c2c(ncn2C)n(C)c1=O]

or a pharmaceutically acceptable salt thereof, wherein one or more hydrogen atoms are optionally replaced with deuterium, for the manufacture of a medicament for treating chronic kidney disease (CKD) in a patient with CKD, wherein the patient's protein to creatinine ratio (PCR) has been assessed and the compound is for administration when the PCR threshold has been assessed to be greater than a threshold, the threshold being greater than 700 mg/g. The PCR has been determined from a urine sample obtained from the patient. In some embodiments, the threshold level is 700 mg/g but not greater than 8500 mg/g. Alternatively, the PCR threshold level is about 700 mg/g, 800 mg/g, 900 mg/g, 1000 mg/g, 1100 mg/g, 1200 mg/g, 1300 mg/g, 1400 mg/g, 1500 mg/g, 1600 mg/g, 1700 mg/g, 1800 mg/g, 1900 mg/g, 2000 mg/g, 2100 mg/g, 2200 mg/g or 2300 mg/g. Alternatively, the PCR threshold level is about 700 mg/g, 800 mg/g, 900 mg/g, 1000 mg/g, 1100 mg/g, 1200 mg/g, 1300 mg/g, 1400 mg/g, 1500 mg/g, 1600 mg/g, 1700 mg/g, 1800 mg/g, 1900 mg/g, 2000 mg/g, 2100 mg/g, 2200 mg/g or 2300 mg/g, but not greater than 8500 mg/g. In some embodiments, the patient also has type 2 diabetes.

Another embodiment of the invention is the use of a therapeutic composition for the manufacture of a medicament for treating chronic kidney disease (CKD) in a patient with CKD, wherein the patient's protein to creatinine ratio (PCR) has been assessed, wherein the composition comprises a compound represented by structural formula (I)

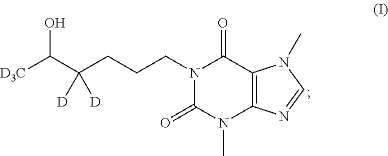 [SMILES: [2H]C([2H])([2H])C(O)C([2H])([2H])CCCn1c(=O)c2c(ncn2C)n(C)c1=O]

or a pharmaceutically acceptable salt thereof, wherein one or more hydrogen atoms are optionally replaced with deuterium, for administration when the PCR threshold has been assessed to be greater than a threshold, the threshold being greater than 700 mg/g and wherein the composition for administration does not comprise a compound represented by structural formula (I) for administration when the PCR threshold has been assessed as less than 700 mg/g (e.g., comprises a drug effective for the treatment of chronic kidney disease other than the compound represented by structural formula (I) or a pharmaceutically acceptable salt thereof). The PCR has been determined from a urine sample obtained from the patient. In some embodiments, the threshold level is 700 mg/g but not greater than 8500 mg/g. Alternatively, the PCR threshold level is about 700 mg/g, 800 mg/g, 900 mg/g, 1000 mg/g, 1100 mg/g, 1200 mg/g, 1300 mg/g, 1400 mg/g, 1500 mg/g, 1600 mg/g, 1700 mg/g, 1800 mg/g, 1900 mg/g, 2000 mg/g, 2100 mg/g, 2200 mg/g or 2300 mg/g. Alternatively, the PCR threshold level is about 700 mg/g, 800 mg/g, 900 mg/g, 1000 mg/g, 1100 mg/g, 1200 mg/g, 1300 mg/g, 1400 mg/g, 1500 mg/g, 1600 mg/g, 1700 mg/g, 1800 mg/g, 1900 mg/g, 2000 mg/g, 2100 mg/g, 2200 mg/g or 2300 mg/g, but not greater than 8500 mg/g. In some embodiments, the patient has type 2 diabetes.

The compounds described have anti-fibrotic effects that are believed to be largely responsible for their ability to protect kidney function. While various biological processes contribute to kidney disease such as inflammation and reactive oxygen species, fibrosis is a final common pathway for kidney failure. In particular, the compound of formula I effectively reduces the levels of certain fibrotic markers including urinary fibronectin and plasma collagen IV. In one embodiment, the present invention provides a method of reducing the level of urinary fibronectin in a patient comprising administering to the patient an effective amount of a compound of structural formula (I) or a pharmaceutically acceptable salt thereof. One aspect of this embodiment relates to a method of reducing the level of urinary fibronectin in a patient with CKD characterized by macroalbuminuria. Another embodiment provides a method of reducing the level of plasma collagen IV in a patient comprising administering to the patient an effective amount of a compound of structural formula (I) or a pharmaceutically acceptable salt thereof. One aspect of this embodiment relates to a method of reducing the level of plasma collagen IV in a patient with CKD characterized by macroalbuminuria. In another aspect, the patient is treated with a drug other than the compound of structural formula (I) if the patient's UACR is below the threshold characterizing macroalbuminuria. In some embodiments, the patient also has type 2 diabetes.

The term “treat” means decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorder that damages or interferes with the normal function of a cell, tissue, or organ.

The term “compound,” when referring to a compound used in the disclosed methods, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The term “isotopologue” refers to a species in which the chemical structure differs from a specific compound of this invention only in the isotopic composition thereof. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound. In the compounds of this invention unless otherwise specified any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

It will be recognized that some variation of natural isotopic abundance occurs in a synthesized compound depending upon the origin of chemical materials used in the synthesis. The concentration of naturally abundant stable hydrogen isotopes, notwithstanding this variation, is small and immaterial as compared to the degree of stable isotopic substitution of compounds of this invention. See, for instance, Wada, E et al., Seikagaku, 1994, 66:15; Gannes, L Z et al., Comp Biochem Physiol Mol Integr Physiol, 1998, 119:725.

The term “isotopic enrichment factor” as used herein means the ratio between the isotopic abundance and the natural abundance of a specified isotope.

In yet another embodiment, a compound described herein (e.g., compounds of structural formula (I), (II), (III), (IV) or (V) or a pharmaceutically acceptable salt thereof) has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Throughout this specification, a variable may be referred to generally (e.g., “each Z”) or may be referred to specifically (e.g., Z³, Z⁴, Z⁵, etc.). Unless otherwise indicated, when a variable is referred to generally, it is meant to include all specific embodiments of that particular variable (for example, “Z” includes Z³, Z⁴ and Z⁵).

In 1^{st} specific embodiment, for the methods, uses, or compositions described above, the compound of structural formula (I) is represented by the following structural formula:

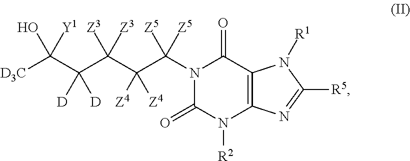

or a pharmaceutically acceptable salt thereof, wherein:
each of R¹ and R² is independently selected from —CH₃ and —CD₃;
R⁵ is hydrogen or deuterium;
each Z³ is hydrogen or deuterium;
each Z⁴ is hydrogen or deuterium;
each Z⁵ is hydrogen or deuterium; and
Y¹ is hydrogen or deuterium.

In one embodiment, for compound of structural formula (II), each Z is hydrogen.

In a 2^{nd} specific embodiment, for methods, uses, or compositions described above, the compound of structural formula (I) is represented by the following structural formula:

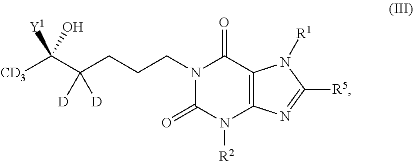

or a pharmaceutically acceptable salt thereof, wherein the variables are as defined above for structural formula (II).

In a 3^{rd} specific embodiment, for methods, uses, or compositions described in the first and second embodiments, the compound of structural formula (I) is represented by the following structural formula:

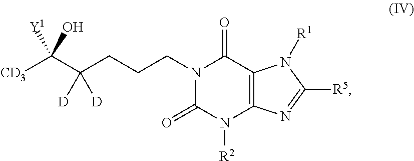

or a pharmaceutically acceptable salt thereof, wherein the variables are as defined above for structural formula (II).

In a 4^{th} specific embodiment, for compounds of structural formula (II), (III) or (IV), R⁵ is deuterium, and the remainder of the variables are as defined in the 1^{st}, 2^{nd} or 3^{rd} specific embodiment.

In a 5^{th} specific embodiment, for compounds of structural formula (II), (III) or (IV), R⁵ is hydrogen, and the remainder of the variables are as defined in the 1^{st}, 2^{nd} or 3^{rd} specific embodiment.

In a 6^{th} specific embodiment, for compounds of structural formula (II), (III) or (IV), R¹ is —CH₃ and R² is —CD₃; and the remainder of the variables are as defined in the 1^{st}, 2^{nd}, 3^{rd}, 4^{th} or 5^{th} specific embodiment.

In a 7^{th} specific embodiment, for compounds of structural formula (II), (III) or (IV), R¹ is —CD₃ and R² is —CH₃; and the remainder of the variables are as defined in the 1^{st}, 2^{nd}, 3^{rd}, 4^{th}, or 5^{th} specific embodiment.

In a 8^{th} specific embodiment, for compounds of structural formula (II), (III) or (IV), R¹ and R² are both —CH₃; and the remainder of the variables are as defined in the 1^{st}, 2^{nd}, 3^{rd}, 4^{th}, or 5^{th} specific embodiment.

In a 9^{th} specific embodiment, for compounds of structural formula (II), (III) or (IV), R¹ and R² are both —CD₃; and the remainder of the variables are as defined in the 1^{st}, 2^{nd}, 3^{rd}, 4^{th}, or 5^{th} specific embodiment.

In a 10^{th} specific embodiment, for compounds of structural formula (II), (III) or (IV), Y¹ is deuterium; and the remainder of the variables are as defined in the 1^{st}, 2^{nd}, 3^{rd}, 4^{th}, 5^{th}, 6^{th}, 7^{th}, 8^{th} or 9^{th} specific embodiment. Alternatively, Y¹ is hydrogen.

In an 11^{th} specific embodiment, for methods, uses, or compositions described herein, the compound is represented by the following structural formula:

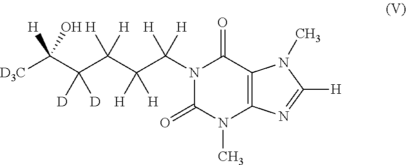 [SMILES: [2H]C([2H])([2H])[C@H](O)C([2H])([2H])CCCn1c(=O)c2c(ncn2C)n(C)c1=O]

or a pharmaceutically acceptable salt thereof.

The invention also provides the use of salts of the compounds described herein.

A salt of a compound of described herein is formed between an acid and a basic group of the compound, such as an amino functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

The compounds described (e.g., compounds of structural formula (I), (II), (III), (IV) or (V), or a pharmaceutically acceptable salt thereof), contain an asymmetric carbon atom. As such, compounds of this invention can exist as either individual enantiomers, or mixtures of the two enantiomers. Accordingly, a compound used in the disclosed methods, uses, or compositions may exist as either a racemic mixture or a scalemic mixture, or as individual respective enantiomer that are substantially free from another possible enantiomer. Unless otherwise indicated, when a disclosed compound is named or depicted by a structure without specifying the stereochemistry, it is understood to represent either a racemic mixture or a scalemic mixture, or each individual enantiomer substantially free from the other enantiomer.

When a particular enantiomer of a compound used in the disclosed methods, uses, or compositions is depicted by name or structure, the enantiomeric purity of the compounds is at least 20%, 30%, 40%, 50%, 60%, 70%, 80%, 90%, 95%, 97%, 99%, 99.5% or 99.9%. “Enantiomeric purity” means the weight percent of the desired enantiomer relative to the combined weight of both enantiomers.

In another embodiment, any atom not designated as deuterium in any of the embodiments set forth above is present at its natural isotopic abundance.

The compounds used in the disclosed methods, uses, or compositions can be prepared by methods disclosed in U.S. Pat. No. 8,263,601 and U.S. Published Application No. 20090239886, the entire teachings of which are incorporated herein by reference.

The term “patient” as used herein includes human or a non-human patients, but is preferably a human.

In another embodiment, for methods, uses, or compositions described herein, the patient is administered an effective amount of a compound described herein. As used herein, the term “effective amount” refers to an amount which, when administered in a proper dosing regimen, is sufficient to therapeutically treat the target disorder. For example, and effective amount is sufficient to reduce or ameliorate the severity, duration or progression of the disorder being treated, slow the advancement of the disorder being treated, cause the regression of the disorder being treated, or enhance or improve the therapeutic effect(s) of another therapy.

The interrelationship of dosages for animals and humans (based on milligrams per meter squared of body surface) is described in Freireich et al., Cancer Chemother. Rep, 1966, 50: 219. Body surface area may be determined approximately from height and weight of the patient. See, e.g., Scientific Tables, Geigy Pharmaceuticals, Ardsley, N.Y., 1970, 537.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician. For example, guidance for selecting an effective dose can be determined by reference to the prescribing information for pentoxifylline.

In one embodiment, for methods described herein, the compound described herein (e.g., compounds of formula (I), (II), (III), (IV) or (V), or a pharmaceutically acceptable salt thereof) is administered to the patient at a dosage range of 300 mg/day to 2400 mg/day, 400 mg/day to 2400 mg/day, 600 mg/day to 2400 mg/day, 600 mg/day to 1800 mg/day, 600 mg/day to 1200 mg/day, 900 mg/day to 2400 mg/day or 900 mg/day to 1800 mg/day. In one embodiment, the patient is administered with the compound at a dosage of 300 mg/day, 400 mg/day, 600 mg/day, 900 mg/day, 1200 mg/day, 1500 mg/day or 1800 mg/day. In one embodiment, any one of these dosages are administered once per day. Alternatively, any one of these dosages are administered twice per day.

In another embodiment, the compound can be administered to the patient once a day, twice a day, or three times a day. In another embodiment, the 600 mg of compound is administered to the patient twice daily.

The present invention also provides a use of a compound described herein (e.g., a compound of structural formula (I), (II), (III), (IV), (V) or a pharmaceutically acceptable salt thereof) in the manufacture of a medicament for treating a disease or disorder described herein (e.g., treating chronic kidney disease) characterized by macroalbuminuria in a patient. In another embodiment, the present invention relates to a use of a compound described herein (e.g., a compound of structural formula (I), (II), (III), (IV), (V) or a pharmaceutically acceptable salt thereof) in the manufacture of a medicament for treating a disease or disorder described herein (e.g., treating chronic kidney disease) characterized by macroalbuminuria in a patient, wherein the patient's UACR is assessed and is found to be greater than a threshold. The UACR threshold level before treatment is, for example, greater than 500 mg/g, 550 mg/g, 600 mg/g, 650 mg/g, 700 mg/g, 750 mg/g, 800 mg/g, 850 mg/g, 900 mg/g, 950 mg/g, 1000 mg/g, 1100 mg/g 1200 mg/g 1300 mg/g, 1400 mg/g or 1500 mg/g. In some embodiments, the UACR threshold level before treatment is, for example, greater than 500 mg/g, 550 mg/g, 600 mg/g, 650 mg/g, 700 mg/g, 750 mg/g, 800 mg/g, 850 mg/g, 900 mg/g, 950 mg/g, 1000 mg/g, 1100 mg/g 1200 mg/g 1300 mg/g, 1400 mg/g or 1500 mg/g, but not greater than 5000 mg/g. In another embodiment, the present invention relates to a use of a compound described herein (e.g., a compound of structural formula (I), (II), (III), (IV), (V) or a pharmaceutically acceptable salt thereof) in the manufacture of a medicament for treating a disease or disorder described herein (e.g., treating chronic kidney disease) characterized by macroalbuminuria in a patient, wherein the patient's PCR is assessed and is found to be greater than a threshold, the threshold level being e.g., 700 mg/g, 800 mg/g, 900 mg/g, 1000 mg/g, 1100 mg/g, 1200 mg/g, 1300 mg/g, 1400 mg/g, 1500 mg/g, 1600 mg/g, 1700 mg/g, 1800 mg/g, 1900 mg/g, 2000 mg/g, 2100 mg/g, 2200 mg/g or 2300 mg/g before the treatment. In some embodiments, the PCR threshold level is 700 mg/g, 800 mg/g, 900 mg/g, 1000 mg/g, 1100 mg/g, 1200 mg/g, 1300 mg/g, 1400 mg/g, 1500 mg/g, 1600 mg/g, 1700 mg/g, 1800 mg/g, 1900 mg/g, 2000 mg/g, 2100 mg/g, 2200 mg/g or 2300 mg/g before the treatment, but not greater than 8500 mg/g.

Another aspect of the invention is a compound described herein (e.g., a compound of structural formula (I), (II), (III), (IV), (V) or a pharmaceutically acceptable salt thereof) for use in the treatment of a disease or disorder described herein (e.g., treating chronic kidney disease) characterized by macroalbuminuria in a patient. In another embodiment, the invention provides a compound described herein (e.g., a compound of structural formula (I), (II), (III), (IV), (V) or a pharmaceutically acceptable salt thereof) for use in the treatment of a disease or disorder thereof described herein (e.g., treating chronic kidney disease) characterized by macroalbuminuria in a patient wherein the patient's UACR is assessed and is found to be greater than a threshold. The UACR threshold level before treatment is, for example, greater than 500 mg/g, 550 mg/g, 600 mg/g, 650 mg/g, 700 mg/g, 750 mg/g, 800 mg/g, 850 mg/g, 900 mg/g, 950 mg/g, 1000 mg/g, 1100 mg/g, 1200 mg/g, 1300 mg/g, 1400 mg/g or 1500 mg/g before treatment. In some embodiments, the UACR threshold level before treatment is, for example, greater than 500 mg/g, 550 mg/g, 600 mg/g, 650 mg/g, 700 mg/g, 750 mg/g, 800 mg/g, 850 mg/g, 900 mg/g, 950 mg/g, 1000 mg/g, 1100 mg/g, 1200 mg/g, 1300 mg/g, 1400 mg/g or 1500 mg/g before treatment, but not greater than 5000 mg/g. In another embodiment, the invention provides a compound described herein (e.g., a compound of structural formula (I), (II), (III), (IV), (V) or a pharmaceutically acceptable salt thereof) for use in the treatment of a disease or disorder thereof described herein (e.g., treating chronic kidney disease) characterized by macroalbuminuria in a patient wherein the patient's PCR is assessed and is found to be greater than a threshold, the threshold level being e.g., 700 mg/g, 800 mg/g, 900 mg/g, 1000 mg/g, 1100 mg/g, 1200 mg/g, 1300 mg/g, 1400 mg/g, 1500 mg/g, 1600 mg/g, 1700 mg/g, 1800 mg/g, 1900 mg/g, 2000 mg/g, 2100 mg/g, 2200 mg/g or 2300 mg/g, before the treatment. In some embodiments, the PCR threshold level is 700 mg/g, 800 mg/g, 900 mg/g, 1000 mg/g, 1100 mg/g, 1200 mg/g, 1300 mg/g, 1400 mg/g, 1500 mg/g, 1600 mg/g, 1700 mg/g, 1800 mg/g, 1900 mg/g, 2000 mg/g, 2100 mg/g, 2200 mg/g or 2300 mg/g, before the treatment, but not greater than 8500 mg/g.

In a further embodiment for the methods described in the previous two paragraphs, an effective amount of a chronic kidney disease therapy that does not include the compound of structural formula (I) or a pharmaceutically acceptable salt thereof if the patients UACR or PCR is below the threshold. In a further embodiment for the methods described in the previous two paragraphs, the patient also has type 2 diabetes.

In one embodiment, any of the above methods of treatment comprises the further step of co-administering to the patient an effective amount of an angiotensin-converting enzyme (ACE) inhibitor and/or an angiotensin receptor blocker (ARB). Specific examples of ACE inhibitors include, but are not limited to, benazepril (Lotensin®), captopril (Capoten®), enalapril (Vasotec®), fosinopril (Monopril®), lisinopril (Prinivil®, Zestril®), moexipril (Univasc®), perindopril (Aceon®), quinapril (Accupril®), ramapril (Altace®), and trandolapril (Mavik®). Specific examples of ARBs include, but are not limited to, candesartan (Atacand®), eprosartan (Teveten®), irbesartan (Avapro®), losartan (Cozaar®), olmesartan (Benicar®), telmisartan (Micardis®) and valsartan (Diovan®).

In yet another embodiment, any of the above methods of treatment comprises the further step of co-administering to the patient an effective amount of second therapeutic agent effective for the treatment of chronic kidney disease (optionally in combination with an effective amount of an angiotensin-converting enzyme (ACE) inhibitor and/or an angiotensin receptor blocker (ARB). Examples of such therapeutic agents include atrasentan, Canagliflozin, and pyridorin. There are a number of other potential second therapeutic agents in phase 2 clinical development.

A “chronic kidney disease therapy that does not include the compound of structural formula (I)” includes administering an effective amount of an ACE inhibitor and/or ARB in the absence of the compound represented by structural formula (I). Alternatively, “chronic kidney disease therapy that does not include the compound of structural formula (I)” includes administering an effective amount of an agent described in the previous paragraph in the absence of the compound represented by structural formula (I). In another alternative, “chronic kidney disease therapy that does not include the compound of structural formula (I)” refers to administering an effective amount of an ACE inhibitor and/or ARB in combination with an effective amount of an agent described in the previous paragraph in the absence of the compound represented by structural formula (I).

The term “co-administered” as used herein means that the second therapeutic agent may be administered together with a compound described herein (e.g., a compound of structural formula (I), (II), (III), (IV), (V) or a pharmaceutically acceptable salt thereof) as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound described herein. In such combination therapy treatment, both the compounds used in the disclosed methods and the second therapeutic agent(s) are administered by conventional methods. The administration of a combination used in the disclosed methods, comprising both a compound of the invention and a second therapeutic agent, to a patient does not preclude the separate administration of that same therapeutic agent, any other second therapeutic agent or any compound of this invention to said patient at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment, for methods described herein, a composition comprising an effective amount of a compound described herein (e.g., compounds of structural formula (I), (II), (III), (IV), (V) or a pharmaceutically acceptable salt thereof) can be used. In one embodiment, the composition is a pharmaceutical compositions comprising an effective amount of a compound described herein (e.g., compounds of structural formula (I), (II), (III), (IV), (V) or a pharmaceutically acceptable salt thereof) and an acceptable carrier.

In one embodiment, the pharmaceutical composition is pyrogen-free. Preferably, a composition of this invention is formulated for pharmaceutical use (“a pharmaceutical composition”), wherein the carrier is a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds described herein in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound described herein optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

In another embodiment, a composition described above further comprises a second therapeutic agent. In another embodiment, the compound described herein and one or more of any of the above-described second therapeutic agents are in separate dosage forms, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

### Clinical Trial

A Phase 2 placebo-controlled clinical trial of Compound (V) was conducted in patients with type 2 diabetic kidney disease and macroalbuminuria. All patients enrolled in the clinical trial were concurrently treated with angiotensin modulators. The purpose of the trial was to compare Compound (V) to placebo in terms of safety and efficacy with respect to measures of renal function such as UACR, serum creatinine levels, and estimated glomerular filtration rate.

The clinical trial consists of three parts, two of which were completed and a third part that is on-going. Part 1 was a double-blind, parallel, two-arm, placebo-controlled study evaluating the safety and efficacy of 600 mg of Compound (V) twice daily for 24 weeks. 182 patients were enrolled in this first part of the trial and 151 patients completed Part 1. Part 2 was a blinded 24-week extension study in which all patients who completed Part 1 were eligible to continue receiving 600 mg of Compound (V) or placebo twice daily. 143 patients were enrolled in this part of the clinical trial and have completed dosing. 123 of the 143 patients that were enrolled in Part 2 of the clinical trial completed Part 2. The combined 48 weeks of data from Parts 1 and 2 of the clinical trial have been analyzed. 102 patients enrolled in Part 3 which is a 48 week open-label extension study that is on-going. All patients who completed Part 2 were eligible to receive 600 mg of Compound (V) twice daily.

The key criteria for patients to be included in the clinical trial were (a) eGFR from 23 to 89 mL/min/1.73 m², a measure of kidney function which indicates mild to moderately severe type 2 diabetic kidney disease; (b) having been on a stable angiotensin modulation regimen for a minimum of four weeks prior to initiating screening and nine weeks prior to initiating dosing; (c) blood pressure less than or equal to 145/90 mm Hg; (d) glycosylated hemoglobin A1c (HbA1c) less than or equal to 10.5%, and (e) UACR greater than or equal to 200 mg/g in male patients and 300 mg/g in female patients, ratios of albumin to creatinine that are indicative of substantial kidney damage in men and women, but not more than 5,000 mg/g, a ratio indicative of severe kidney disease.

Preliminary data analyzed to date from the first 48 weeks of treatment supports the potential of Compound (V) to help protect kidney function in patients at risk for chronic kidney disease progression. The preliminary 48 week analyses suggested that the serum creatinine levels of patients who received Compound (V) rose less than those of patients who received placebo. Serum creatinine is waste product that is cleared by the kidneys and increasing serum creatinine levels are believed to indicate worsening of kidney function. The preliminary analyses indicate that the mean serum creatinine level in the 65 patients receiving Compound (V) increased by 0.13 mg/dL over the 48 weeks of treatment, as compared to an increase of 0.21 mg/dL in the 58 patients receiving placebo. The lower value in the case of Compound (V) represents a 38% improvement as compared to placebo (p=0.057 using a two-tailed statistical analysis) at 48 weeks and may indicate a slower decline of kidney function in patients treated with Compound (V) than those who received placebo.

In addition, a preliminary post-hoc analysis also indicated a statistically significant effect, at 48 weeks, in reduced incidence of large increases in serum creatinine levels in patients receiving Compound (V) as compared to placebo. After 48 weeks, six out of the 58 patients receiving placebo, or 10.3%, experienced a 50% or greater increase in serum creatinine levels, compared with one out of the 65 patients receiving Compound (V), or 1.5% (p=0.026) in the overall population. After 48 weeks, 5 out of the 27 patients receiving placebo, or 18.5%, experienced a 50% or greater increase in serum creatinine levels, compared with one out of the 31 patients receiving Compound (V), or 3.1% (p<0.0409) in the subpopulation having UACR greater than 850 mg/g. A 50% increase in serum creatinine levels corresponds mathematically to between a 30% and a 40% decline in eGFR.

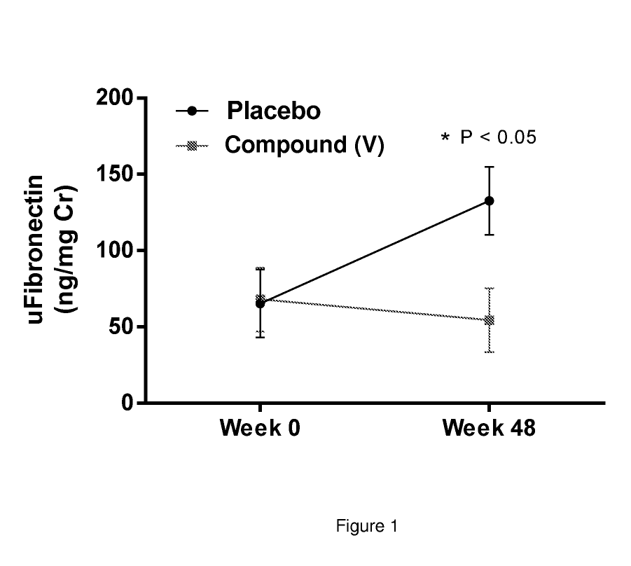

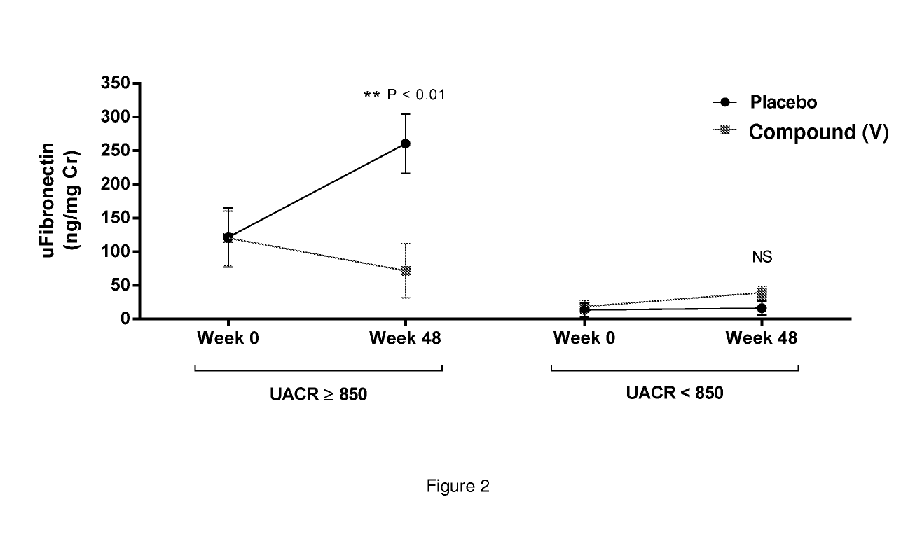

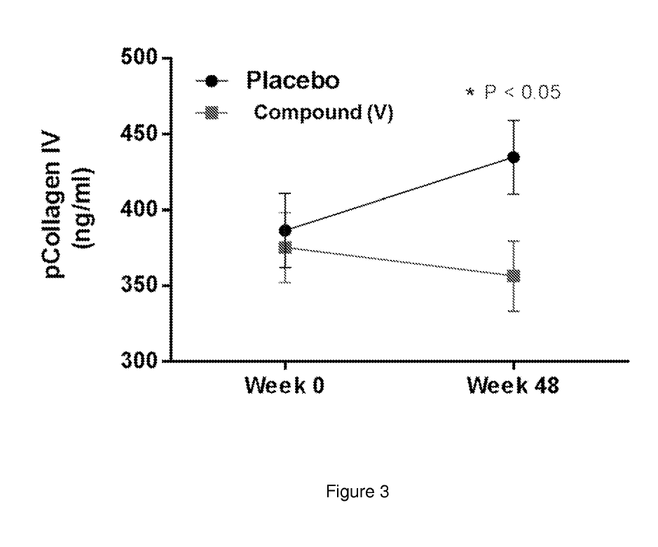

Preliminary analysis of the 48 week clinical trial data also indicated that, relative to placebo, Compound V improved the levels of certain fibrotic biomarkers such as urinary fibronectin and plasma collagen IV. Urinary fibronectin increases in patients with kidney damage due to leakage of the protein into the urine. After 48 weeks, Compound (V)-treated patients had 59% less urine fibronectin at week 48 versus placebo in the overall population (FIG. 1). In the subpopulation having UACR greater than 850 mg/g, the decrease relative to placebo was 73% (p<0.01) (FIG. 2). Compound (V)-treated patients have 18% less plasma collagen IV at week 48 versus placebo (FIG. 3).

What is claimed is:

1. A method of treating chronic kidney disease (CKD) in a patient with CKD characterized by macroalbuminuria with a urine albumin to creatinine ratio (UACR) that is greater than a threshold, the threshold being 500 mg/g before the treatment, comprising administering to the patient an effective amount of a compound represented by structural formula (I): 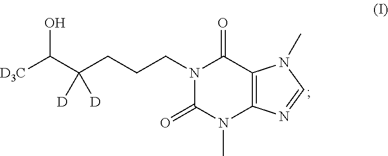 [SMILES: [2H]C([2H])([2H])C(O)C([2H])([2H])CCCn1c(=O)c2c(ncn2C)n(C)c1=O]
or a pharmaceutically acceptable salt thereof, wherein one or more hydrogen atoms are optionally replaced with deuterium.

2. The method of claim 1, wherein the macroalbuminuria is characterized by a urine albumin to creatinine ratio (UACR) that is greater than a threshold, the threshold level being 850 mg/g before the treatment.

3. The method of claim 1, wherein the macroalbuminuria is characterized by a urine albumin to creatinine ratio (UACR) that is greater than a threshold, the threshold level being 1000 mg/g before the treatment.

4. A method of treating chronic kidney disease (CKD) in a patient with CKD characterized by macroalbuminuria comprising the steps of:
(a) assessing the patient's urine albumin to creatinine ratio (UACR); and
(b) if the patient's UACR is greater than a threshold, the threshold level being 500 mg/g, administering to the patient an effective amount of a compound represented by structural formula (I): 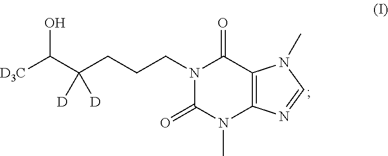 [SMILES: [2H]C([2H])([2H])C(O)C([2H])([2H])CCCn1c(=O)c2c(ncn2C)n(C)c1=O]
or a pharmaceutically acceptable salt thereof, wherein one or more hydrogen atoms are optionally replaced with deuterium.

5. The method of claim 4, wherein the UACR threshold level is 850 mg/g.

6. A method of treating chronic kidney disease (CKD) in a patient with CKD characterized by macroalbuminuria comprising the steps of:
(a) assessing the patient's urine albumin to creatinine ratio (UACR);
(b) if the patient's UACR is greater than a threshold, the threshold level being 500 mg/g, administering to the patient an effective amount of a compound represented by structural formula (I): 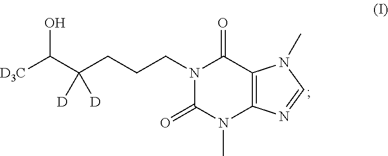 [SMILES: [2H]C([2H])([2H])C(O)C([2H])([2H])CCCn1c(=O)c2c(ncn2C)n(C)c1=O]
or a pharmaceutically acceptable salt thereof, wherein one or more hydrogen atoms are optionally replaced with deuterium; and
c) if the patient's UACR is less than the threshold, the threshold level being 500 mg/g, administering to the patient an effective amount of a chronic kidney disease therapy that does not include the compound of structural formula (I) or a pharmaceutically acceptable salt thereof.

7. The method of claim 6, wherein the UACR threshold is 850 mg/g.

8. The method of claim 1, wherein the compound of structural formula (I) is administered at a dosage range of 600 mg/day to 2400 mg/day.

9. The method of claim 8, wherein the compound structural formula (I) is administered at a dosage range of 600 mg/day to 1800 mg/day.

10. The method of claim 1, wherein the compound is represented by the following structural formula: 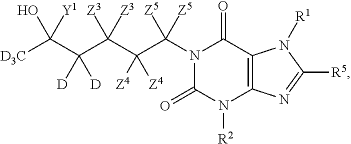
or a pharmaceutically acceptable salt thereof, wherein:
each of R¹ and R² is independently selected from —CH₃ and —CD₃;
R⁵ is hydrogen or deuterium;
each Z³ is hydrogen or deuterium;
each Z⁴ is hydrogen or deuterium;
each Z⁵ is hydrogen or deuterium; and
Y¹ is hydrogen or deuterium.

11. The method of claim 10, wherein each Z is hydrogen.

12. The method of claim 11, wherein the compound is represented by the following structural formula: 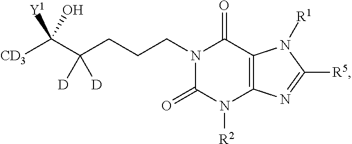
or a pharmaceutically acceptable salt thereof.

13. The method of claim 12, wherein the compound is 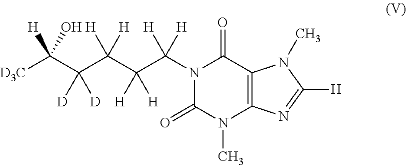 [SMILES: [2H]C([2H])([2H])[C@H](O)C([2H])([2H])CCCn1c(=O)c2c(ncn2C)n(C)c1=O]
or a pharmaceutically acceptable salt thereof.

14. The method of claim 13, wherein the optical purity of the compound is at least 95%.

15. The method of claim 1, wherein any hydrogen atom not designated as deuterium is present at its natural isotopic abundance.

16. The method of claim 1, wherein the isotopic enrichment factor for each designated deuterium atom is at least 6000.

17. The method of claim 1, wherein the isotopic enrichment factor for each designated deuterium atom is at least 6600.

18.-21. (canceled)
